(19) United States
(12) Patent Application Publication
(10) Pub. No.: US 2008/0207882 A1
(43) Pub. Date: Aug. 28, 2008

(54) METHOD FOR PRODUCING THIOPHENE GLYCOSIDE DERIVATIVES

(75) Inventors: Volker DERDAU, Frankfurt (DE); Lars BIERER, Liederbach (DE); Michael KOSSENJANS, Goteborg (SE)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS DEUTSCHLAND GMBH, Frankfurt am Main (DE)

(21) Appl. No.: 11/765,600

(22) Filed: Jun. 20, 2007

Related U.S. Application Data
(63) Continuation of application No. PCT/EP2005/013158, filed on Dec. 8, 2005.

(30) Foreign Application Priority Data
Dec. 22, 2004 (DE) 102004063099.2

(51) Int. Cl. C07H 17/00; C07H 1/00

(52) U.S. Cl. 536/18.2; 536/18.6

(57) ABSTRACT

The present invention relates to a process for preparing thiophene-glycoside derivatives of the general formula (I)

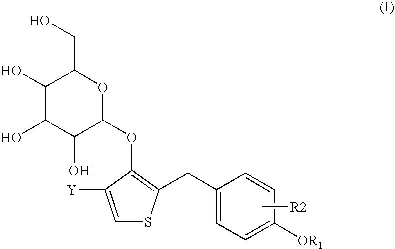

### FIELD OF THE INVENTION

The present invention relates to a process for preparing thiophene-glycoside derivatives of the general formula (I)

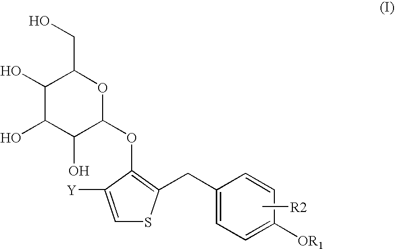

### BACKGROUND OF THE INVENTION

Thiophene-glycoside derivatives show biological activity which makes use possible, in particular, in the prevention and treatment of type 1 and 2 diabetes.

WO2004/007517 describes, inter alia, various processes for preparing thiophene-glycoside derivatives of the general formula (I). However, the most efficient and shortest described process (B) has various disadvantages in relation to an industrial conversion. Thus, the products are purified mainly by chromatography. The yields are moreover so low in some cases that removal of the precursors and by-products impedes simple isolation of the product. No optimization was undertaken in relation to atom economy. The use of highly toxic compounds, such as sodium cyanoborohydride, or substances with a very intense odor, such as dimethyl sulfide, furthermore impair use thereof in an industrial process.

In view of the disadvantages and problems described above, there is a need to provide a process which avoids these disadvantages and problems and which moreover, without requiring great additional complexity, can be implemented in a simple manner and makes the desired products available in high yields with high conversion and high selectivity. High yields, in particular, are a central requirement for an improved process.

### SUMMARY OF THE INVENTION

This object is surprisingly achieved, in accordance with the present invention, by a process for preparing compounds of the general formula (I):

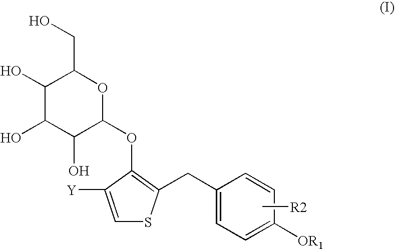

in which:
Y is H, or (C₁-C₁₀)-alkyl;
R₁ is (C₁-C₈)-alkyl, where one, more than one or all hydrogen(s) may be replaced by fluorine; (C₅-C₁₀)-aryl, where aryl may also comprise 1 to 3 heteroatoms selected from O, N, and S;
R₂ is H, Cl, Br, or I;
   said process comprising multiple stages in which

### A. Preparation of the Hydroxy Ketones

### A.1. A Thiophene Component of the Formula (II)

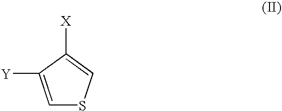

in which Y is as defined above, and
X is O—(C₁-C₈)-alkyl or O—(C₅-C₁₀)-aryl, where aryl may also comprise 1 to 3 heteroatoms selected from O, N, and S;
is reacted with a compound of the formula (III)

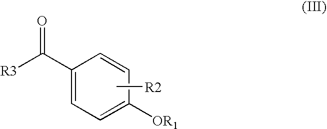

in which
R1 and R2 are as defined above, and

### R3 is Cl, Br, or I;

in the presence of from 0.1 to 10 equivalents, preferably 0.8 to 1.5 equivalents, of one or more acids—one acid being preferred—preferably with a Lewis acid such as SnCl₄, AlCl₃, TiCl₄, BF₃, FeCl₃, ZnCl₂, MgCl₂, ZnBr₂, and MgBr₂—but also with Brönsted acids such as CF₃SO₃H, H₂SO₄, and toluenesulfonic acid, particularly preferably with Lewis acids, for example, SnCl₄ or AlCl₃, in a suitable solvent, preferably in a halogenated solvent such as, for example, dichloromethane, chloroform, or 1,2-dichloroethane, at from about −50° C. to +150° C., preferably at from about −20° C. to +80° C., particularly preferably at from about 50° C. to 25° C., to give a compound of the formula (IV),

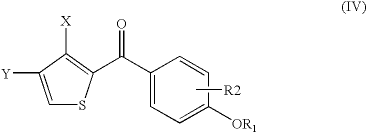

in which X, Y, R1 and R2 are as defined above; and
the compound of the formula (IV)
is then converted, in the presence of from 0.1 to 10 equivalents, preferably 0.8 to 1.5 equivalents, of one or more acids—where one acid is preferred—preferably a Lewis acid such as BBr₃, BCl₃, BF₃, AlCl₃, SnCl₄, or TiCl₄ at from about −50° C. to +150° C., preferably from about −20° C. to +80° C., particularly preferably at from about 0° C. to 25° C., into a compound of the formula (IVa)

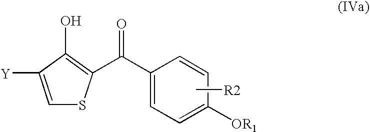

in which Y, R1 and R2 are as defined above,
or

### A.2. The Thiophene Component of the Formula (II)

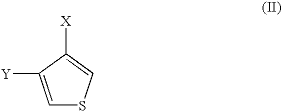

in which X and Y are as defined above under A.1.
is reacted with a compound of the formula (III)

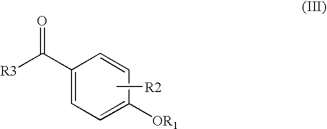

in which
R1, R2 and R3 are as defined above under A.1.;
in the presence of from 0.1 to 10 equivalents, preferably 0.8 to 1.5 equivalents, of one or more acids—one acid being preferred—preferably with a Lewis acid such as SnCl₄, AlCl₃, TiCl₄, BF₃, FeCl₃, ZnCl₂, MgCl₂ ZnBr₂, or MgBr₂—but also Brönsted acids such as CF₃SO₃H, H₂SO₄, or toluenesulfonic acid, particularly preferably with a Lewis acid such as SnCl₄ or AlCl₃, in a suitable solvent, preferably in a halogenated solvent such as, for example, dichloromethane, chloroform, or 1,2-dichloroethane, at from about −50° C. to +150° C., preferably at from about −20° C. to +100° C., particularly preferably at from about 60° C. to 75° C., to give a compound of the formula (IV)

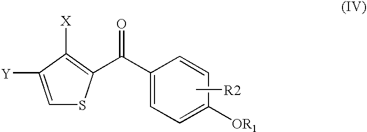

in which X, Y, R1 and R2 are as defined above, and the latter is directly reacted further in the presence of an acid as defined above at from about 0 to 200° C. preferably at from about 20° C. to 120° C., particularly preferably at from about 80 to 90° C., to give the compound of the formula (IVa)

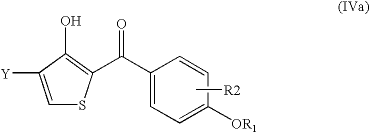

in which Y, R1 and R2 are as defined above, or

### A.3. The Thiophene Component of the Formula (II)

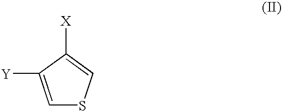

in which X and Y are as defined above,
is reacted with one or more organometallic reagents from the series M-(C₁-C₈)-alkyl, MH, M-O—(C₁-C₈)-alkyl or M-N((C₁-C₈)-alkyl)₂ in which M is Li, Na, K, Zn, Mg, or Ca, in one or more apolar solvents, such as an ether, for example, diethyl ether, tetrahydrofuran, dibutyl ether, dihexyl ether and methyl tert-butyl ether, at temperatures of from about −20° C. to 45° C., preferably at temperatures of from about 15° C. to 35° C., particularly preferably of from about 30° C. to 35° C. to give the reactive intermediate of the formula (V)

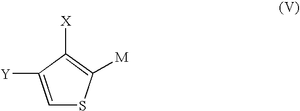

in which X, Y and M are as defined above,
and the latter is reacted further with a compound of the formula (IIIa)

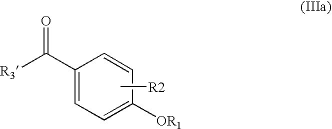

in which R1 and R2 are as defined above, and
R3′ is selected from Cl, Br, I, NH—(C₁-C₈)-alkyl, NH—O—(C₁-C₈)-alkyl, N((C₁-C₈)-alkyl)₂, N—(C₁-C₈)-alkyl-O—(C₁-C₈)-alkyl, N(C₃-C₈)-cycloalkyl, where the alkyl ring may comprise one or more heteroatoms selected from N, O, and S, N((C₆-C₁₀)-aryl)-(C₁-C₈)-alkyl, N((C₃-C₈)-cycloalkyl)-(C₃-C₈)-aryl, and N((C₆-C₁₀)-aryl)₂, where the aromatic systems and the cyclic alkanes may comprise one or more heteroatoms from the series N, O, S,
to give a compound of the formula (IV)

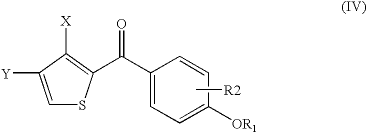

in which X, Y, R1 and R2 are as defined above; as described under A.1. at temperatures of from about −20° C. to +30° C., preferably about 5° C. to +5° C.;
and, subsequently, the compound of the formula (IV) is converted in the presence of a Lewis acid, such as BBr₃, AlCl₃, SnCl₄, or TiCl₄ at from about 0° C. to 30° C., preferably at from about 5° C. to 15° C.,
to a compound of the formula (IVa)

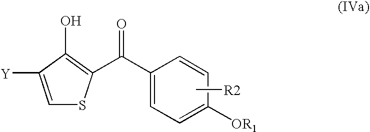

in which Y, R1 and R2 are as defined above;
and, where appropriate, subsequently, the compounds of the formula (IVa) are purified by conventional purification methods such as crystallization, distillation or chromatography, preferably by crystallization from a solvent or a mixture of a plurality of solvents such as alkanes, aromatic compounds, halogenated solvents, ethers, ketones, esters, alcohols or water, particularly preferably purified by crystallization from methanol or from dichloromethane/heptane or methanol/water mixtures or by sodium salt and—after neutralization—crystallization from water;
and, subsequently,

### B. Preparation of the Acetogluco Ketones

a compound of the formula (IVa)

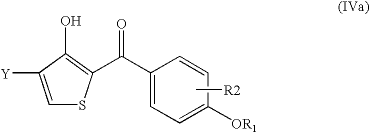

is reacted with from about 0.5 to 10 equivalents, preferably about 1 to 4 equivalents, particularly preferably about 1.5 to 2.0 equivalents, of a sugar derivative of the formula (VI)

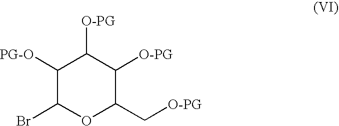

in which PG is an OH protective group such as, for example, methyl, methoxymethyl (MOM), methylthiomethyl (MTM), phenyldimethylsilylmethoxymethyl (SMOM), benzyloxymethyl (BOM), p-methoxybenzyloxymethyl (PMBM), t-butoxymethyl, 4-pentenyloxymethyl, 2-methoxyethoxymethyl (MEM), 2-trimethylsilylethoxymethyl (SEM), trimethylsilyl (TMS), tert-butyldimethylsilyl (TBDMS), tert-butyldiphenylsilyl (TBDPS), triisopropylsilyl (TIPS), or similar silyl protective groups, 1-methyl-1-methoxyethyl (MIP), allyl, benzyl, acetyl, trifluoroacetyl, Fmoc, or THP, preferably acetyl,
in the presence of from 1 to 15 equivalents, preferably 3 to 6 equivalents, of an organic or inorganic base, preferably potassium carbonate, and from 0.01 to 5 equivalents, preferably 0.1 to 1 equivalents, particularly preferably 0.3 to 0.6 equivalents, of a phase transfer catalyst, preferably tetrabutylammonium bromide or chloride or benzyltributylammonium chloride or bromide, in a mixture of an organic solvent, preferably methylene chloride or 2-methyltetrahydrofuran, and water in the ratio of from 10,000:1 to 1:1, preferably 500:1 to 10:1, particularly preferably 200:1 to 50:1, at from about −20° C. to +80° C., preferably at from about 5° C. to 40° C., particularly preferably at from about 20° C. to 30° C., to give a compound of the formula (VII);

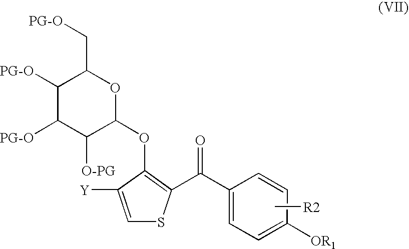

in which PG, Y, R1 and R2 are as defined above;
and, subsequently,

### C. Preparation of the Acetoglucomethylenes

a compound of the formula (VII) as described above is reacted
in a suitable organic solvent such as, for example, dichloromethane, acetonitrile, tetrahydrofuran, dimethylformamide, DMSO or chloroform, preferably in acetonitrile, with from 1 to 15 equivalents, preferably 2 to 6 equivalents, of one or more hydride donors such as, for example, potassium borohydride, sodium borohydride, sodium cyanoborohydride, triethylsilane, and triacetoxyborohydride, preferably with sodium cyanoborohydride or sodium borohydride, particularly preferably with sodium borohydride, and from 0.1 to 5 equivalents, preferably 0.5 to 1.5 equivalents, of one or more activators selected from the group consisting of lithium chloride, bromine, sodium bromide, potassium bromide, iodine, sodium iodide, potassium iodide, sodium triiodide or potassium triiodide, preferably with iodine, and from 1 to 25 equivalents, preferably 3 to 10 equivalents, of one or more further acids, preferably Lewis acids or acid equivalents, such as, for example, trifluoroacetic acid, hydrogen chloride, BF₃, halosilanes, preferably chlorosilanes, particularly preferably trimethylsilyl chloride, at from about −100° C. to +100° C., preferably at from about −40° C. to +40° C., particularly preferably from about −15° C. to +15° C.,
to give a compound of the formula (VIII),

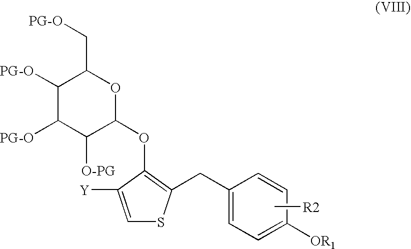

in which PG, Y, R1 and R2 are as defined above; and, subsequently

### D. Preparation of the Thiophene-Glycoside Derivatives

the protective groups are eliminated under basic or acidic conditions, by oxidation or reduction or with fluoride, in accordance with known methods as described, for example, in T. W. Greene, P. Wuts, Protective Groups in Organic Synthesis 1999, Wiley, New York; preferably, as described above, with PG=acetyl in the presence of from 0.01 to 25 equivalents, preferably 0.05 to 5 equivalents, particularly preferably 0.1 to 0.5 equivalents, of an organic or inorganic base, such as, for example, sodium methanolate or potassium methanolate, sodium hydroxide or potassium hydroxide, preferably, sodium methanolate, in a suitable solvent, preferably, methanol, at from about −50° C. to +150° C., preferably at from about −20° C. to +80° C., particularly preferably about 0° C. to 50° C.; and, thereby converted into compounds of the formula (I)

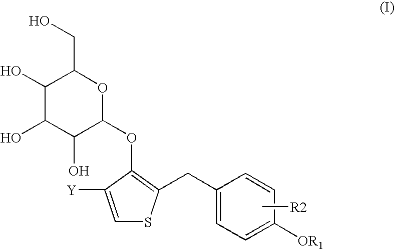

in which Y, R1 and R2 are as defined above;
subsequently, the compounds of the formula (I) are purified by conventional purification methods such as crystallization or chromatography, preferably by crystallization from a solvent or a mixture of a plurality of solvents such as alkanes, aromatic compounds, halogenated solvents, ethers, ketones, esters, alcohols or water, particularly preferably by crystallization from alcohols or alcohols/water mixtures, very particularly preferably by crystallization from methanol/water.

### DETAILED DESCRIPTION

Preference is given to a multistage process for preparing the compounds of the formula (I), in which step A. Preparation of the hydroxy ketones consists of variants A2 or A3 described above:

Process for preparing compounds of the general formula (I):

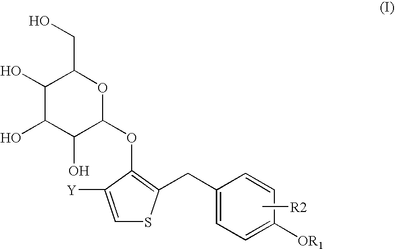

in which the meanings are
Y H, (C₁-C₁₀)-alkyl;
R1 (C₁-C₈)-alkyl, where one, more than one or all hydrogen(s) may be replaced by fluorine; (C₅-C₁₀)-aryl, where aryl may also comprise 1 to 3 heteroatoms from the series O, N, S;
R2H, Cl, Br, I;
   which comprises

### A. Preparation of the Hydroxy Ketones

### A.2. The Thiophene Component of the Formula (II),

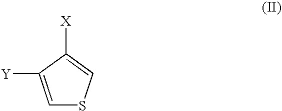

in which Y is as defined above, and
X is O—(C₁-C₈)-alkyl or O—(C₅-C₁₀)-aryl, where aryl may also comprise 1 to 3 heteroatoms from the series O, N, S;
being reacted with a compound of the formula (III)

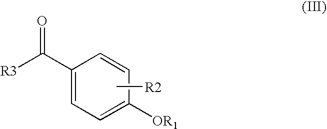

in which
R1 and R2 are as defined above, and

### R3 is Cl, Br, I;

in the presence of from 0.1 to 10 equivalents of one or more acids in a suitable solvent at from −50 to +150° C. to give a compound of the formula (IV)

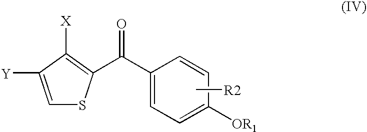

in which X, Y, R1 and R2 are as defined above; and
the latter being directly converted further in the presence of an acid as defined above at from 0 to 200° C. into the compound of the formula (IVa)

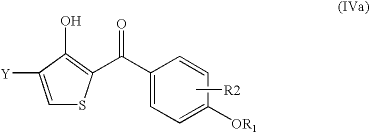

in which Y, R1 and R2 are as defined above,
or

### A.3. The Thiophene Component of the Formula (II)

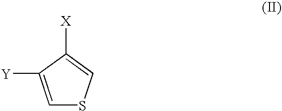

in which X and Y are as defined above,
being reacted with one or more organometallic reagents from the series M-(C₁-C₈)-alkyl, MH, M-O—(C₁-C₈)-alkyl or M-N((C₁-C₈)-alkyl)₂, in which M is Li, Na, K, Zn, Mg, Ca,
in apolar solvents at temperatures of from −20 to 45° C. to give the reactive intermediate of the formula (V)

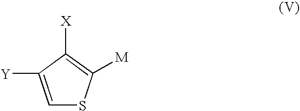

in which X, Y and M are as defined above,
and the latter being reacted further with a compound of the formula (IIIa)

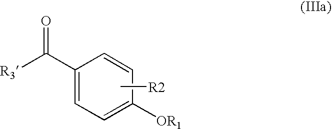

in which R1 and R2 are as defined above, and
R3′ is Cl, Br, I,
   NH—(C₁-C₈)-alkyl, NH—O—(C₁-C₈)-alkyl, N((C₁-C₈)-alkyl)₂, N—(C₁-C₈)-alkyl-O—(C₁-C₈)-alkyl,
   N(C₃-C₈)-cycloalkyl, where the alkyl ring may comprise one or more heteroatoms from the series N, O, S,
   N((C₆-C₁₀)-aryl)-(C₁-C₈)-alkyl, N((C₃-C₈)-cycloalkyl)-(C₃-C₈)-aryl, N((C₆-C₁₀)-aryl)₂, where the aromatic systems and the cyclic alkanes may comprise one or more heteroatoms from the series N, O, S,
      to give a compound of the formula (IV),

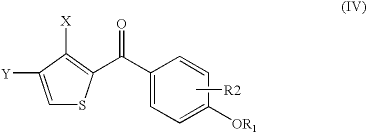

in which X, Y, R1 and R2 are as defined above; as described under A.2. at temperatures of from −20° C. to +30° C.;
and subsequently this compound of the formula (IV) being converted in the presence of a Lewis acid into the compound of the formula (IVa)

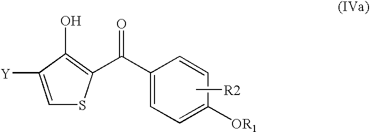

in which Y, R1 and R2 are as defined above, and where appropriate subsequently the compounds of the formula (IVa) being purified by conventional purification methods;
and subsequently

### B. Preparation of the Acetogluco Ketones

the compound of the formula (IVa)

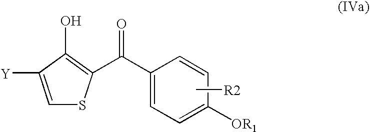

being reacted with from 0.5 to 10 equivalents of a sugar derivative of the formula (VI)

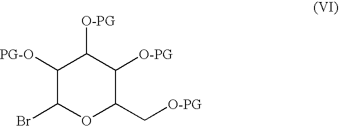

in which PG is an OH protective group in the presence of from 1 to 15 equivalents of an organic or inorganic base and from 0.01 to 5 equivalents of a phase-transfer catalyst in a mixture of an organic solvent and water in the ratio of 10 000:1 to 1:1 at from −20° C. to +80° C. to give the compound of the formula (VII);

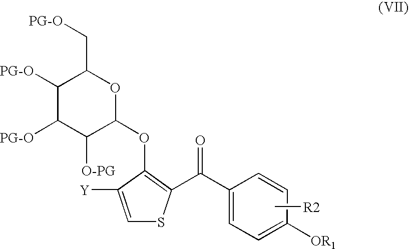

in which PG, Y, R1 and R2 are as defined above;
and subsequently

### C. Preparation of the Acetoglucomethylenes

the compound of the formula (VII) as described above being reacted
in an organic suitable solvent with from 1 to 15 equivalents of one or more hydride donors and from 0.1 to 5 equivalents of one or more activators selected from the group of lithium chloride, bromine, sodium bromide or potassium bromide, iodine, sodium iodide or potassium iodide, sodium triiodide or potassium triiodide, preferably with iodine and from 1 to 25 equivalents of one or more further acids at from −100° C. to +100° C. to give the compound of the formula (VIII)

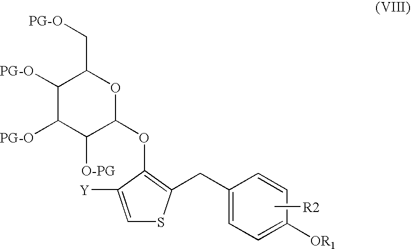

in which PG, Y, R1 and R2 are as defined above;
subsequently

### D. Preparation of the Thiophene-Glycoside Derivatives

the protective groups being eliminated under basic or acidic conditions, by oxidation or reduction or with fluoride, in accordance with known methods, in the presence of from 0.01 to 25 equivalents of an organic or inorganic base in a suitable solvent at from −50° C. to +150° C. and subsequently
being converted into the compounds of the formula (I)

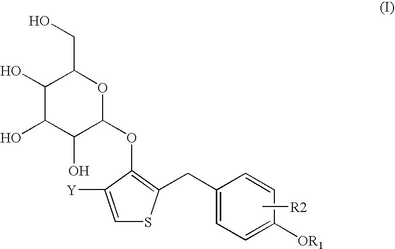

in which Y, R1 and R2 are as defined above,
and subsequently the compounds of the formula (I) being purified by conventional purification methods.

The invention also relates to a process for preparing the intermediate compounds of the formula (VIII), in which a compound of the formula (VII)

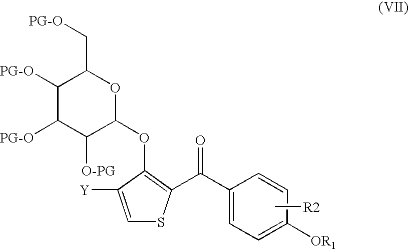

in which
PG is an OH protective group;
Y is H, (C₁-C₁₀)-alkyl;
R1 is (C₁-C₈)-alkyl, where one, more than one or all hydrogen(s) may be replaced by fluorine; (C₅-C₁₀)-aryl, where aryl may also comprise 1 to 3 heteroatoms from the series O, N, S;
R2H, Cl, Br, I;
   is reacted in an organic suitable solvent with from 1 to 15 equivalents of one or more hydride donors and from 0.1 to 5 equivalents of one or more activators selected from the group of lithium chloride, bromine, sodium bromide or potassium bromide, iodine, sodium iodide or potassium iodide, sodium triiodide or potassium triiodide and from 1 to 25 equivalents of one or more further acids at from −100° C. to +100° C. to give the compound of the formula (VIII)

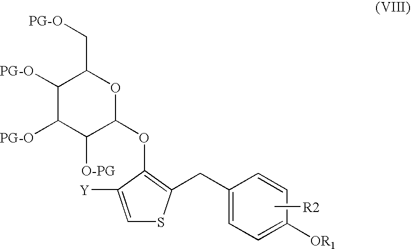

in which PG, Y, R1 and R2 are as defined above.

In a preferred process for preparing the intermediate compounds of the formula (VIII), iodine is used as activator.

A further preferred embodiment is a process for preparing the compounds of the formula (I) in which the meanings are
Y H;
R1 (C₁-C₄)-alkyl, where one, more than one or all hydrogen(s) may be replaced by fluorine, preferably CH₃, C₂H₅, CF₃;
R2 H.

The invention relates to compounds of the formula (I) in the form of their racemates, racemic mixtures and pure enantiomers, to their diastereomers and mixtures thereof, and the alkali metal, alkaline earth metal, ammonium, iron and similar pharmacologically acceptable salts thereof.

The alkyl radicals, including alkoxy, alkenyl and alkynyl, in the substituents R1, R3′, X, Y and M may be either straight-chain or branched.

The sugar residues in the compounds of the formula (I) represent both L- and D-sugars in their alpha(α) and beta(β) forms, such as, for example, allose, altrose, glucose, mannose, gulose, idose, galactose, talose. Those which may be mentioned as preferred are: D-glucose, D-galactose, D-allose and D-mannose, particularly preferably β-D-glucose and β-D-galactose, very particularly preferably β-D-glucose.

The process of the invention is notable in particular for making an industrially feasible route possible to thiophene-glycoside derivatives in high yields. The alternative processes for preparing the compound (IV) provide the option of employing a large number of acid- or base-labile precursors of the compound (III).

The following examples illustrate the process without restricting it:

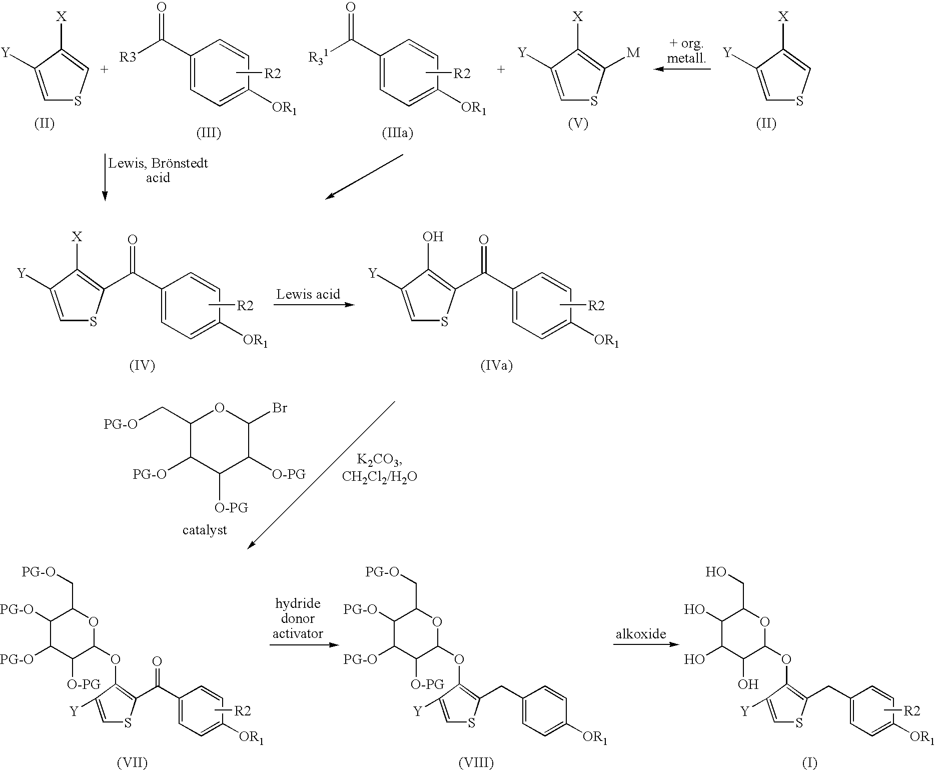

### EXAMPLE 1

### a) (4-Methoxyphenyl)(3-methoxythiophen-2-yl)methanone (Variant A1)

24.4 parts by weight of tin tetrachloride are dissolved in 300 parts by volume of dichloromethane in a reaction vessel and, at an internal temperature of 5-10° C., 15.0 parts by weight of p-anisoyl chloride are added. Then 9.56 parts by weight of 3-methoxythiophene are added at an internal temperature of 5-10° C., and the reaction mixture is stirred at 20-25° C. for 3-5 h. After conversion is complete (check of conversion), 135 parts by volume of water are added to the reaction mixture. It is then washed with 25 parts by volume of 30% strength hydrochloric acid. The organic and aqueous phase are separated, and the organic phase is washed with 100 parts by volume of water, 100 parts by volume of 8% strength sodium bicarbonate solution and 100 parts by volume of water. The organic phase is concentrated by distillation to 40 parts by volume and, at 40° C., 210 parts by volume of heptane are metered in. The suspension is cooled to 0° C., and the solid is freed of solvent. The pale yellow solid is then dried. The product is obtained in 94% yield; m.p. 98-99° C., ¹H-NMR (CDCl₃): d=8.37 (d, J=6.3 Hz, 1H), 7.96 (d, J=6.9 Hz, 2H), 6.96 (d, J=6.9 Hz, 2H), 6.37 (d, J=6.3 Hz, 1H), 3.91, 3.88 (s, 6H) ppm.

### b) (3-Hydroxythiophen-2-yl-(4-methoxyphenyl)methanone

1.86 parts by weight of boron tribromide are added to a solution of 1.84 parts by weight of (4-methoxyphenyl)(3-methoxythiophen-2-yl)methanone in 25 parts by volume of dichloromethane at 0-5° C., and the mixture is stirred at 5-15° C. for 60 min. It is then stirred at 20-25° C. for a further 3 h, and then 1.0 parts by volume of methanol and 12 parts by volume of water are added. A pH of 8 is adjusted with about 1.4 parts by volume of 33% strength sodium hydroxide solution. The phases are separated, and the organic phase is washed twice with 10 parts by volume of water each time. The organic phase is concentrated in vacuo, and the residue is taken up in 20 parts by volume of methanol. The solution is heated to 60° C., and 4 parts by volume of water are added. After cooling to 0° C., the precipitated solid is separated off and dried. The product is obtained as a dark gray solid in 91% yield; m.p.: 86-87° C. ¹H-NMR (DMSO-d₆): δ=11.85 (s, 1H, OH), 7.96 (d, J=5.4 Hz, 1H), 7.89 (d, J=8.8 Hz, 2H), 7.09 (d, J=8.8 Hz, 2H), 6.91 (d, J=5.4 Hz, 1H), 3.85 (s, 3H) ppm.

### EXAMPLE 2

### (3-Hydroxythiophen-2-yl)(4-trifluoromethoxyphenyl)methanone (Variant A2)

0.86 parts by weight of 4-trifluoromethoxybenzoyl chloride are added to a solution of 1.0 parts by weight of tin tetrachloride in 10.8 parts by volume of 1,2-dichloroethane. The solution is heated to 68-70° C. and, at this temperature, 0.4 part by weight of 3-methoxythiophene are added over 2 h. The reaction mixture is refluxed at 70° C. for 3 h (check of conversion to (IV)) and for a further 8 h (80-85° C., check of conversion to (IVa)). At 25° C., 3.7 parts by weight of water and 6.3 parts by volume of 30% strength hydrochloric acid are added. After addition of 24 parts by volume of heptane, the phases are separated, and the organic phase is washed with 10 parts by volume of deionized water. The solvent is concentrated to 16 parts by volume. Filtration and washing with heptane are carried out. The filtrate is stirred with 25 parts by volume of 0.8% strength sodium hydroxide solution, and the phases are separated. The aqueous phase is washed with heptane. A pH of 9.0 is adjusted with 7.5% strength hydrochloric acid, whereupon the product precipitates again. The product is filtered off with suction, washed and dried (3-hydroxythiophen-2-yl)(4-trifluoromethoxyphenyl)methanone is isolated as brownish to yellowish solid in 53% yield. m.p.: 67-70° C.; ¹H-NMR (DMSO-d₆): δ=11.45 (br s, 1H, OH), 7.97 (d, J=5.4 Hz, 1H), 7.93 (d, J=8.7 Hz, 2H), 7.51 (d, J=8.7 Hz, 2H), 6.87 (d, J=5.4 Hz, 1H) ppm.

### EXAMPLE 3

### a) (4-Trifluoromethoxyphenyl)-(3-methoxythiophen-2-yl)methanone (Variant A3)

8 parts by volume of n-BuLi (1.6 M in hexane) are added to 7 parts by volume of 3-methoxythiophene in 150 parts by volume of diethyl ether at 20-25° C. under a protective gas atmosphere, and the solution is heated at 40° C. for 30 min. The reaction mixture is added to an ice-cooled solution (0-5° C.) of 8.3 parts by weight of N-methoxy-N-methyl-4-trifluoromethoxybenzamide in 100 parts by volume of diethyl ether. The mixture is then stirred at room temperature for 1 h (check of conversion). 50 parts by volume of water are added, the phases are separated and the aqueous phase is extracted 3× with dichloromethane, the combined organic phases are dried over Na₂SO₄, and the solvent is removed in vacuo. 76% of the product are isolated as a yellowish oil. ¹H-NMR (DMSO-d₆): δ=8.04 (d, J=5.5 Hz, 1H), 7.82 (d, J=8.6 Hz, 2H), 7.45 (d, J=8.6 Hz, 2H), 7.19 (d, J=5.5 Hz, 1H), 3.79 (s, 3H) ppm.

### b) (3-Hydroxythiophen-2-yl)(4-trifluoromethoxyphenyl)methanone

7.56 parts by weight of (3-methoxythiophen-2-yl)(4-trifluoromethoxyphenyl)-methanone in 100 parts by volume of dichloromethane are slowly added to a solution of 8.2 parts by weight of BBr₃×DMS in 500 parts by volume of dichloromethane at 20-25° C. The dark solution is stirred at 20-25° C. for 7 h (check of conversion) and then 80 parts by volume of saturated sodium bicarbonate solution are added in one portion. The phases are separated, the organic phase is washed with 100 parts by volume of water and dried, and the solvent is removed in vacuo. The solid is recrystallized in methanol, and 86% of a pale yellow solid are obtained.

### EXAMPLE 4

### 4,5-Diacetoxy-6-acetoxymethyl-2-[2-(4-methoxybenzoyl)thiophen-3-yloxy]tetrahydropyran-3-yl acetate

3.9 parts by weight of benzyltributylammonium chloride, 19.4 parts by weight of potassium carbonate and 2.6 parts by volume of water are added to a solution of 7.3 parts by weight of (3-hydroxythiophen-2-yl)(4-methoxyphenyl)methanone in 280 parts by volume of dichloromethane at 20-25° C. Over the course of 2 h, 22.5 parts by weight of 2,3,4,6-tetra-O-acetyl-alpha-D-glucopyranosyl bromide are added. The reaction mixture is stirred at 20-25° C. for 16 h (check of conversion), solids are removed and the organic phase is washed 3× with water. The organic phase is concentrated and taken up in 95 parts by volume of methanol. After crystallization, the solution is cooled to 0° C. The solid is separated off and dried. 81% of the product are obtained as a colorless solid; m.p.: 149-151° C., ¹H-NMR (DMSO-d₆): δ=8.0 (d, 1H), 7.7 (d, 2H), 7:1 (d, 2H), 7.0 (d, 1H), 5.6 (d, 1H), 5.3 (dd, 1H), 4.9 (m, 1H), 4.7 (dd, 1H), 4.2 (m, 2H), 4.1 (m, 1H), 3.8. (s, 3H, O—CH3), 2.05, 2.00, 1.90, 1.85 (s, 12H, acetyl-CH3) ppm.

### EXAMPLE 5

### 4,5-Diacetoxy-6-acetoxymethyl-2-[2-(4-trifluoromethoxybenzoyl)thiophen-3-yloxy]-tetrahydropyran-3-yl acetate

3.5 parts by weight of benzyltributylammonium chloride, 15.3 parts by weight of potassium carbonate and 2.5 parts by volume of water are added to a solution of 7.1 parts by weight of (3-hydroxythiophen-2-yl)(4-trifluoromethoxyphenyl)methanone in 250 parts by volume of dichloromethane at 20-25° C. Over the course of 2 h, 18.7 parts by weight of 2,3,4,6-tetra-O-acetyl-alpha-D-glucopyranosyl bromide are added. The reaction mixture is stirred at 20-25° C. for 16 h (check of conversion), solids are removed and the organic phase is washed 3× with water. The organic phase is concentrated and taken up in 100 parts by volume of isopropanol. At 40-45° C., 75 parts by weight of water are added, and the solution is cooled to 0° C. The solid is separated off and dried. 90% of the product are obtained as a colorless solid; m.p.: 90-93° C., ¹H-NMR (DMSO-d6): δ=8.09 (d, J=5.5 Hz, 1H), 7.78 (d, J=6.7 Hz, 2H), 7.43 (d, J=6.7 Hz, 2H), 7.13 (d, J=5.5 Hz, 1H), 5.60 (d, J=7.9 Hz, 1H), 5.27 (dd, J=9.5/9.5 Hz, 1H), 4.94-4.90 (m, 1H), 4.63 (dd, J=9.6/9.5 Hz, 1H), 4.21-4.17 (m, 2H), 4.06-4.04 (m, 1H), 2.02, 1.99, 1.90, 1.84 (s, 12H, acetyl-CH3) ppm.

### EXAMPLE 6

### 4,5-Diacetoxy-6-acetoxymethyl-2-[2-(4-methoxybenzyl)thiophen-3-yloxy]tetrahydropyran-3-yl acetate

4.5 parts by weight of iodine and 2.1 parts by weight of sodium borohydride (added over 60 min), and 11.5 parts by weight of trimethylsilyl chloride (added over 45 min) are added to a solution of 10.3 parts by weight of 4,5-diacetoxy-6-acetoxymethyl-2-[2-(4-methoxybenzoyl)thiophen-3-yloxy]tetrahydropyran-3-yl acetate in 57 parts by weight of acetonitrile at from −10 to 0° C. After being stirred at 0° C. for 90 min, the reaction mixture is diluted with 75 parts by volume of dichloromethane and, while cooling, 75 parts by volume of water are added dropwise. After washing with water several times, the solvent is removed in vacuo, and the residue in 51 parts by volume of methanol. The crude product is recrystallized at 50-60° C. and then filtered off with suction at −5° C. The colorless solid is dried and obtained in a yield of 83%. m.p.: 116-118° C.; ¹H-NMR (DMSO-d₆): δ=7.29 (d, J=5.5 Hz, 1H), 7.09 (d, J=6.7 Hz, 2H), 6.87 (d, J=5.5 Hz, 1H), 6.84 (d, J=6.7 Hz, 2H), 5.41-5.33 (m, 2H), 5.07-4.97 (m, 2H), 4.21-4.17 (m, 2H), 4.09 (d, J=9.7 Hz, 1H), 3.91-3.79 (m, 2H), 3.71 (s, 3H), 2.00, 1.99, 1.96, 1.95 (s, 12H, acetyl-CH₃) ppm.

### EXAMPLE 7

### 4,5-Diacetoxy-6-acetoxymethyl-2-[2-(4-trifluoromethoxybenzyl)thiophen-3-yloxy]-tetrahydropyran-3-yl acetate

3.24 parts by weight of iodine and 2.0 parts by weight of sodium borohydride (added over 60 min), and 11.1 parts by weight of trimethylsilyl chloride (added over 45 min) are added to a solution of 7.98 parts by weight of 4,5-diacetoxy-6-acetoxymethyl-2-[2-(4-trifluoromethoxybenzoyl)thiophen-3-yloxy]tetrahydropyran-3-yl acetate in 41.6 parts by weight of acetonitrile at from −10 to 0° C. After stirring at 0° C. for 90 min, the reaction mixture is diluted with 77 parts by volume of dichloromethane and, while cooling, 77 parts by volume of water are added dropwise. After washing with water several times, the solvent is removed in vacuo and the residue is taken up in 35 parts by volume of methanol. The crude product is recrystallized at 40-50° C. and then filtered off with suction at −10° C. The colorless solid is dried and 81% of a colorless solid are obtained. m.p.: 113-114° C.; ¹H-NMR (DMSO-d₆): δ=7.47 (d, J=8.1 Hz, 2H), 7.40 (d, J=5.5 Hz, 1H), 7.30 (d, J=8.1 Hz, 2H), 6.86 (d, J=5.5 Hz, 1H), 5.89 (d, J=3.6 Hz, 1H), 5.45 (dd, J=9.8/9.3 Hz, 1H), 5.38 (d, J=8.0 Hz, 1H), 5.11 (dd, J=8.0/9.8 Hz, 1H), 5.04 (dd, J=9.3/9.3 Hz, 1H), 4.21-4.17 (m, 2H), 4.10 (dd, J=5.0/9.8 Hz, 1H), 3.33 (s, 2H), 2.09, 2.01, 2.00, 1.99 (s, 12H, acetyl-CH₃) ¹³C-NMR (DMSO-d₆): δ=170.0, 169.6, 169.3, 169.3, 148.9, 147.2, 144.1, 129.5, 127.4, 123.8, 120.7, 118.9, 99.6, 71.8, 70.9, 70.8, 68.1, 66.1, 61.7, 20.4, 20.4, 20.3, 20.3 ppm.

### EXAMPLE 8

### 2-Hydroxymethyl-6-[2-(4-methoxybenzyl)thiophen-3-yloxy]tetrahydropyran-3,4,5-triol

0.97 parts by weight of sodium methanolate (30% in methanol) are added to a suspension of 14.5 parts by weight of 4,5-diacetoxy-6-acetoxymethyl-2-[2-(4-methoxybenzyl)thiophen-3-yloxy]tetrahydropyran-3-yl acetate in 91 parts by weight of methanol at 0° C. The reaction mixture is stirred at 0° C. for 90 min and then a pH of 7 is adjusted with 0.76 parts by weight of acetic acid. The product is precipitated by adding water and is filtered off with suction at 0° C. The colorless solid is dried and obtained in a yield of 83%. m.p.: 154-155° C.; ¹H-NMR (DMSO-d₆): δ=7.16-7.14 (m, 3H), 6.91 (d, J=5.5 Hz, 1H), 6.80 (d, J=8.6 Hz, 2H), 5.35 (s, 1H), 5.05 (s, 1H), 4.99 (s, 1H), 4.63-4.53 (m, 2H), 4.01-3.97 (m, 2H), 3.71 (s, 3H), 3.66 (s, 1H), 3.49-3.44 (m, 1H), 3.32-3.05 (m, 4H) ppm.

### EXAMPLE 9

### 2-Hydroxymethyl-6-[2-(4-trifluoromethoxybenzyl)thiophen-3-yloxy]tetrahydropyran-3,4,5-triol

1.5 parts by weight of sodium methanolate (30% in methanol) are added to a suspension of 12.3 parts by weight of 4,5-diacetoxy-6-acetoxymethyl-2-[2-(4-trifluoromethoxybenzyl)thiophen-3-yloxy]tetrahydro-pyran-3-yl acetate in 83.2 parts by weight of methanol at 0° C. The reaction mixture is stirred at 10° C. for 90 min and then a pH of 7 is adjusted with 1.58 parts by weight of acetic acid. The product is precipitated by adding water and is filtered off with suction at 0° C. The colorless solid is dried and obtained in a yield of 89%. m.p.: 144-145° C.; ¹H-NMR (DMSO-d₆): δ=7.41 (d, J=8.5 Hz, 2H), 7.27 (d, J=8.5 Hz, 2H), 7.24 (d, J=5.5 Hz, 1H), 6.97 (d, J=5.5 Hz, 1H), 5.37 (d, J=4.9 Hz, 1H), 5.05 (d, J=4.5 Hz, 1H), 4.98 (d, J=5.3 Hz, 1H), 4.64 (d, J=7.3 Hz, 1H), 4.56 (dd, J=5.7/5.7 Hz, 1H), 4.12-4.04 (m, 2H), 3.72-3.68 (m, 1H), 3.51-3.47 (m, 1H), 3.32-3.12 (m, 4H); ¹⁹F-NMR (DMSO-d₆): δ=56.8 ppm.

1. A process for preparing a compound of the general formula (I): 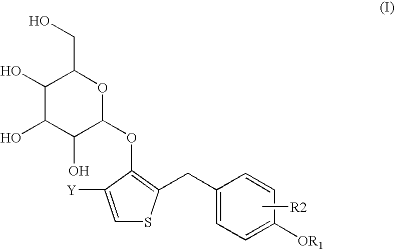
in which:
Y is H, (C₁-C₁₀)-alkyl;
R1 is (C₁-C₈)-alkyl, where one, more than one or all hydrogen(s) may be replaced by fluorine; (C₅-C₁₀)-aryl, where aryl may also comprise 1 to 3 heteroatoms selected from O, N, and S; and
R2 is H, Cl, Br, or I;
which comprises reacting a thiophene component of the formula (II) 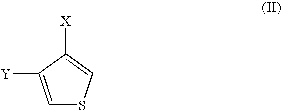
in which Y is as defined above, and
X is O—(C₁-C₈)-alkyl or O—(C₅-C₁₀)-aryl, where aryl may also comprise 1 to 3 heteroatoms selected from O, N, and S;
with a compound of the formula (III) 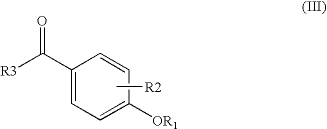
in which
R1 and R2 are as defined above, and
R3 is Cl, Br, or I;
in the presence of from 0.1 to 10 equivalents of one or more acids in a suitable solvent at a temperature of from about −50 to about +150° C. to give a compound of formula (IV), 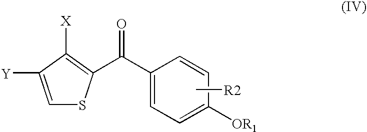
in which X, Y, R1 and R2 are as defined above; and then converting the compound of formula (IV), in the presence of from about 0.1 to 10 equivalents of one or more acids at from about −50 to about +150° C. into a compound of formula (IVa) 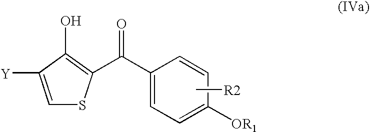
in which Y, R1 and R2 are as defined above;
or, in the alternative, reacting a thiophene component of formula (II) 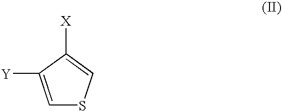
in which X and Y are as defined above with a compound of formula (III) 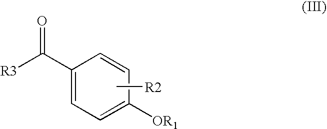
in which
R1, R2 and R3 are as defined above;
in the presence of from 0.1 to 10 equivalents of one or more acids in a suitable solvent at from about −50 to about +150° C. to give a compound of formula (IV) 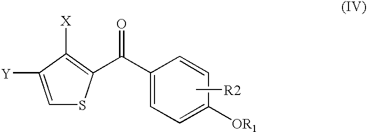
in which X, Y, R1 and R2 are as defined above; and then directly further reacting said compound of formula (IV) in the presence of an acid as defined above at from about 0 to about 200° C. to give a compound of formula (IVa) 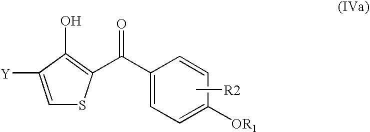
in which Y, R1 and R2 are as defined above,
or, alternatively, reacting the thiophene component of formula (II) 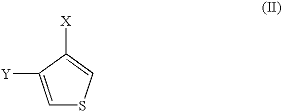
in which X and Y are as defined above,
with one or more organometallic reagents selected from M-(C₁-C₈)-alkyl, MH, M-O—(C₁-C₈)-alkyl and M-N((C₁-C₈)-alkyl)₂ in which M is Li, Na, K, Zn, Mg, or Ca, in an apolar solvent at a temperature of from about −20 to about 45° C. to give the reactive intermediate of formula (V) 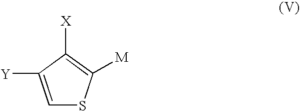
in which X, Y and M are as defined above,
and then reacting said intermediate of formula (V) further with a compound of formula (IIIa) 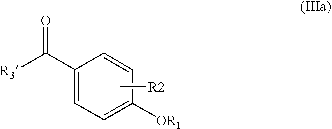
in which R1 and R2 are as defined above, and
R3′ is selected from Cl, Br, I, NH—(C₁-C₈)-alkyl, NH—O—(C₁-C₈)-alkyl, N((C₁-C₈)-alkyl)₂, N—(C₁-C₈)-alkyl-O—(C₁-C₈)-alkyl, and N(C₃-C₈)-cycloalkyl, where the alkyl ring may comprise one or more heteroatoms selected from N, O, and S, as well as from N((C₆-C₁₀)-aryl)-(C₁-C₈)-alkyl, N((C₃-C₈)-cycloalkyl)-(C₃-C₈)-aryl, N((C₆-C₁₀)-aryl)₂, where the aromatic systems and the cyclic alkanes may comprise one or more heteroatoms selected from N, O, and S,
to give a compound of formula (IV) 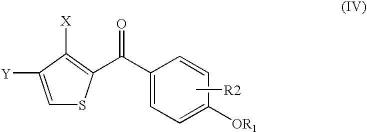
in which X, Y, R1 and R2 are as defined above; at a temperature of from about −20° C. to about +30° C.;
and subsequently converting said compound of formula (IV) in the presence of a Lewis acid, into a compound of formula (IVa) 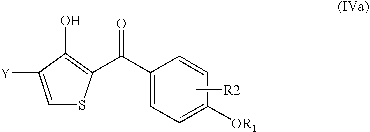
in which Y, R1 and R2 are as defined above, and, where appropriate, subsequently purifying said compound of formula (IVa) by a conventional purification method; and subsequently reacting said compound of formula (IVa) 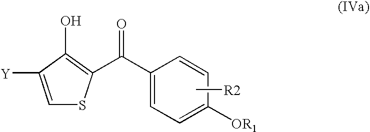
with from about 0.5 to about 10 equivalents of a sugar derivative of formula (VI) 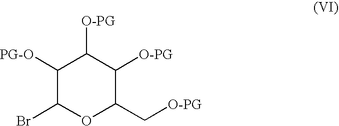
in which PG is an OH protective group, in the presence of from about 1 to about 15 equivalents of an organic or inorganic base and from about 0.01 to about 5 equivalents of a phase-transfer catalyst in a mixture of an organic solvent and water in a ratio of from about 10 000:1 to about 1:1 at a temperature of from about −20° C. to about +80° C. to give a compound of formula (VII); 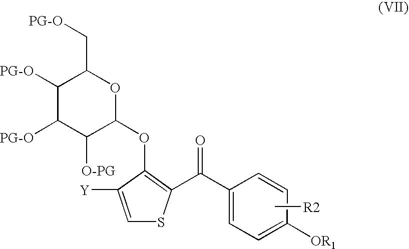
in which PG, Y, R1 and R2 are as defined above;
and subsequently reacting said compound of formula (VII), in a suitable organic solvent with from about 1 to about 15 equivalents of one or more hydride donors and from about 0.1 to about 5 equivalents of one or more activators selected from the group consisting of lithium chloride, bromine, sodium bromide, potassium bromide, iodine, sodium iodide, potassium iodide, sodium triiodide and potassium triiodide and from about 1 to about 25 equivalents of one or more further acids at a temperature of from about −100° C. to about +100° C. to give a compound of formula (VIII) 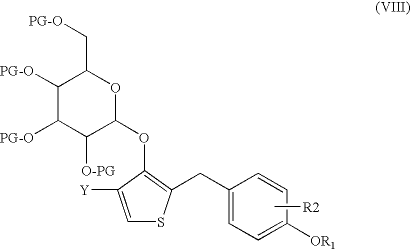
in which PG, Y, R1 and R2 are as defined above; and, subsequently, eliminating the protective groups under basic or acidic conditions, by oxidation or reduction or with fluoride, using known methods, in the presence of from about 0.01 to about 25 equivalents of an organic or inorganic base in a suitable solvent at a temperature of from about −50° C. to about +150° C. and subsequently converting into a compound of formula (I) 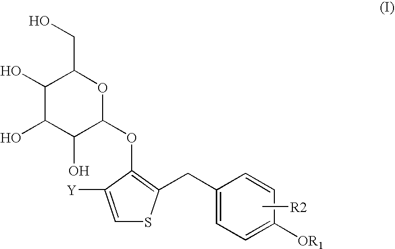
in which Y, R1 and R2 are as defined above,
and thereafter purifying said compound of formula (I) by a conventional purification method.

2. A process for preparing a compound of general formula (I): 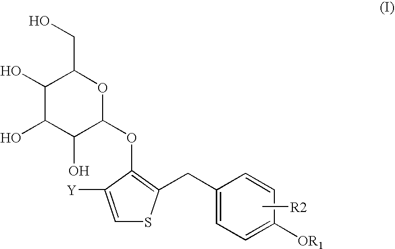
in which:
Y is H or (C₁-C₁₀)-alkyl;
R1 is either (C₁-C₈)-alkyl, where one, more than one or all hydrogen(s) may be replaced by fluorine; or (C₅-C₁₀)-aryl, where aryl may also comprise 1 to 3 heteroatoms selected from O, N, and S;
R2 is H, Cl, Br, or I; which comprises reacting a thiophene component of formula (II) 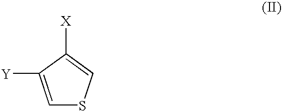
in which Y is as defined above, and
X is O—(C₁-C₈)-alkyl or O—(C₅-C₁₀)-aryl, where aryl may also comprise 1 to 3 heteroatoms selected from O, N, and S;
with a compound of formula (III) 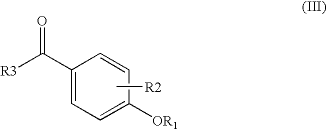
in which
R1 and R2 are as defined above, and
R3 is Cl, Br, or I;
in the presence of from about 0.1 to about 10 equivalents of one or more acids in a suitable solvent at from about −50 to about +150° C. to give a compound of formula (IV), 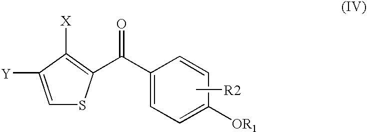
in which X, Y, R1 and R2 are as defined above; and directly converting said compound of formula (IV), in the presence of an acid as defined above, at a temperature of from about 0 to about 200° C. into a compound of formula (IVa) 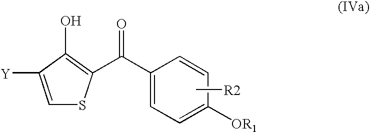
in which Y, R1 and R2 are as defined above, or, alternatively, reacting a thiophene component of formula (II) 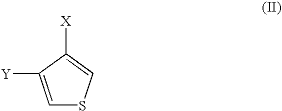
in which X and Y are as defined above, with one or more organometallic reagents selected from M-(C₁-C₈)-alkyl, MH, M-O—(C₁-C₈)-alkyl or M-N((C₁-C₈)-alkyl)₂ in which M is Li, Na, K, Zn, Mg, or Ca,
in an apolar solvent at a temperature of from about −20 to about 45° C. to give a reactive intermediate of formula (V) 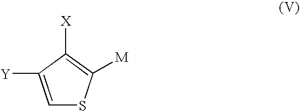
in which X, Y and M are as defined above,
and then further reacting said intermediate (V) with a compound of formula (IIIa) 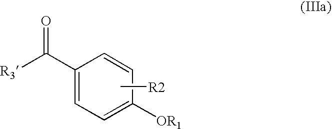
in which R1 and R2 are as defined above, and
R3′ is selected from Cl, Br, I, NH—(C₁-C₈)-alkyl, NH—O—(C₁-C₈)-alkyl, N((C₁-C₈)-alkyl)₂, N—(C₁-C₈)-alkyl-O—(C₁-C₈)-alkyl, and N(C₃-C₈)-cycloalkyl, where the alkyl ring may comprise one or more heteroatoms selected from N, O, and S, as well as from N((C₆-C₁₀)-aryl)-(C₁-C₈)-alkyl, N((C₃-C₈)-cycloalkyl)-(C₃-C₈)-aryl, and N((C₆-C₁₀)-aryl)₂, where the aromatic systems and the cyclic alkanes may comprise one or more heteroatoms selected from N, O, and S,
to give a compound of formula (IV) 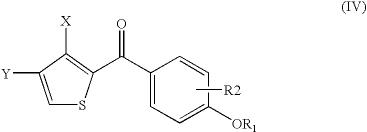
in which X, Y, R1 and R2 are as defined above, at a temperature of from about −20° C. to about +30° C.; and subsequently converting said compound of formula (IV), in the presence of a Lewis acid, into a compound of formula (IVa) 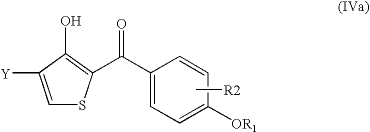
in which Y, R1 and R2 are as defined above, and, where appropriate, subsequently purifying said compound of formula (IVa) by a conventional purification method;
and subsequently reacting said compound of formula (IVa) 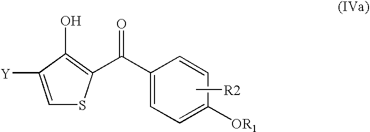
with from about 0.5 to about 10 equivalents of a sugar derivative of formula (VI) 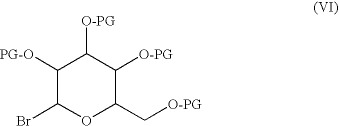
in which PG is an OH protective group, in the presence of from about 1 to about 15 equivalents of an organic or inorganic base and of from about 0.01 to about 5 equivalents of a phase-transfer catalyst in a mixture of an organic solvent and water in the ratio of from about 10,000:1 to about 1:1 at a temperature of from about −20° C. to about +80° C. to give a compound of the formula (VII); 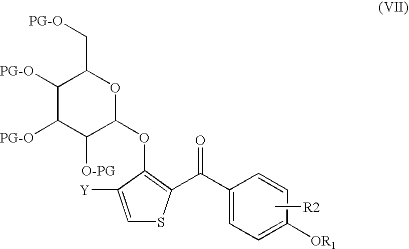
in which PG, Y, R1 and R2 are as defined above;
and, subsequently, reacting the compound of the formula (VII) as described in a suitable organic solvent with from about 1 to about 15 equivalents of one or more hydride donors and from about 0.1 to about 5 equivalents of one or more activators selected from the group consisting of lithium chloride, bromine, sodium bromide, potassium bromide, iodine, sodium iodide, potassium iodide, sodium triiodide and potassium triiodide and from about 1 to about 25 equivalents of one or more further acids at from about −100° C. to about +100° C. to give a compound of the formula (VIII) 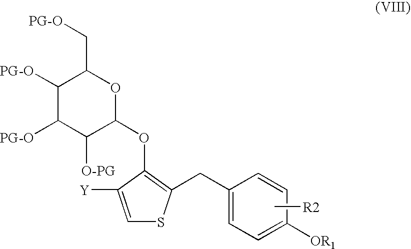
in which PG, Y, R1 and R2 are as defined above; and,
subsequently, eliminating the protective groups under basic or acidic conditions, by oxidation or reduction or with fluoride, using known methods, in the presence of from about 0.01 to about 25 equivalents of an organic or inorganic base in a suitable solvent at from about −50° C. to about +150° C. and, subsequently, converting the resulting product into said compound of formula (I) 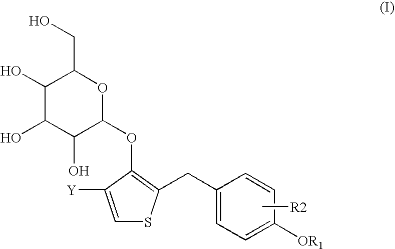
in which Y, R1 and R2 are as defined above,
and subsequently purifying said compound of formula (I) by conventional purification methods.

3. The process as claimed in claim 1, wherein said activator is iodine.

4. The process as claimed in claim 2, wherein said activator is iodine

5. A process for preparing an intermediate compound of formula (VIII), which comprises reacting a compound of formula (VII), 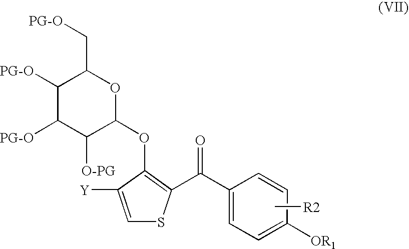
in which
PG is an OH protective group;
Y is H, or (C₁-C₁₀)-alkyl;
R1 is (C₁-C₈)-alkyl, where one, more than one or all hydrogen(s) may be replaced by fluorine; or (C₅-C₁₀)-aryl, where aryl may also comprise 1 to 3 heteroatoms selected from O, N, and S; and
R2 is H, Cl, Br, or I;
in a suitable organic solvent, with from about 1 to about 15 equivalents of one or more hydride donors and from about 0.1 to about 5 equivalents of one or more activators selected from the group consisting of lithium chloride, bromine, sodium bromide, potassium bromide, iodine, sodium iodide, potassium iodide, sodium triiodide and potassium triiodide and with from about 1 to about 25 equivalents of one or more further acids at from about −100° C. to about +100° C. to give said compound of the formula (VIII) 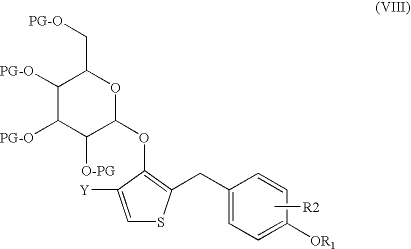
in which PG, Y, R1 and R2 are as defined above.

6. The process as claimed in claim 5, wherein the activator is iodine.

7. The process as claimed in claim 1, wherein
Y is H;
R1 is (C₁-C₄)-alkyl, where one, more than one or all hydrogen(s) may be replaced by fluorine; and
R2 is H.

8. The process as claimed in claim 2, wherein
Y is H;
R1 is (C₁-C₄)-alkyl, where one, more than one or all hydrogen(s) may be replaced by fluorine; and
R2 is H.

9. The process as claimed in claim 3, wherein
Y is H;
R1 is (C₁-C₄)-alkyl, where one, more than one or all hydrogen(s) may be replaced by fluorine; and
R2 is H.
